(19) United States
(12) Patent Application Publication
(10) Pub. No.: US 2023/0012443 A1
(43) Pub. Date: Jan. 12, 2023

(54) MOUNTING STRUCTURE FOR ENDOSCOPE FRONT-END PIECE

(71) Applicant: JOYMEDICARE (SHANGHAI) MEDICAL ELECTRONIC TECHNOLOGY CO., LTD, Shanghai (CN)

(72) Inventors: Liang GU, Shanghai (CN); Suyun LI, Shanghai (CN)

(73) Assignee: JOYMEDICARE (SHANGHAI) MEDICAL ELECTRONIC TECHNOLOGY CO., LTD, Shanghai (CN)

(21) Appl. No.: 17/933,489

(22) Filed: Sep. 20, 2022

Related U.S. Application Data
(63) Continuation of application No. PCT/CN2021/072723, filed on Jan. 19, 2021.

(30) Foreign Application Priority Data
May 25, 2020 (CN) 202010449920.7
May 25, 2020 (CN) 202020895196.6

(51) Int. Cl. A61B 1/005; A61B 1/00

(52) U.S. Cl. CPC A61B 1/0055; A61B 1/00112

(57) ABSTRACT

The present disclosure provides a mounting structure for an endoscope front-end piece, including a front-end piece, a pull rod, and an adjustment mechanism. The front-end piece includes a front end clamped onto a port of an endoscope tube, and a rear end extended into the endoscope tube; a sealant groove is formed in a portion of the front-end piece contacting an inner wall of the endoscope tube; the pull rod includes a front end connected to the front-end piece, and a rear end extended into an endoscope tube socket; and the adjustment mechanism is located in the endoscope tube socket, and is capable of both compressing the endoscope tube socket and tensioning the pull rod through threaded transmission, thereby allowing the front-end piece to retain on the port of the endoscope tube, and adjusting a mounting gap and a mounting pressure. The structure realizes gapless mounting of the front-end piece.

### CROSS-REFERENCE TO RELATED APPLICATIONS

This application is a continuation of International Patent Application No. PCT/CN2021/072723 with a filing date of Jan. 19, 2021, designating the United States, now pending, and further claims priority to Chinese Patent Application No. 202010449920.7 with a filing date of May 25, 2020 and Chinese Patent Application No. 202020895196.6 with a filing date of May 25, 2020. The content of the aforementioned applications, including any intervening amendments thereto, are incorporated herein by reference.

### TECHNICAL FIELD

The present disclosure belongs to the field of endoscopes, and particularly relates to a mounting structure for an endoscope front-end piece.

### BACKGROUND

An endoscope is generally composed of an operation portion and an insertion portion. The insertion portion is an elongated tube, through which various tubes pass to realize functions of the endoscope. A part at a frontmost end of the insertion portion refers to an endoscope front-end piece, a function of which is to seal a cavity of the endoscope to protect an internal structure thereof in most cases. Typically, there are some through holes that are formed to accommodate an endoscope imaging system, a surgical operating instrument, etc. The endoscope front-end piece comes into direct contact with objects. Particularly, the medical endoscope front-end piece contacts human tissues and organs, and needs to be disinfected and sterilized at a high temperature and a high pressure after use. Hence, higher requirements are put forward for the quality, machining precision and mounting reliability of parts on the endoscope front-end piece.

For existing endoscopes, the endoscope front-end piece is mainly welded or glued with an endoscope tube. However, due to specificity of the welding process, the welded endoscope front-end piece tends to be structurally simple, and can only take a protective function. Meanwhile, the assembly of the whole product is restricted seriously, and the endoscope can only be assembled from a rear end to a front end with a cannula and the like, such as a structure of a front-end cover on an insertion portion in the Chinese Patent Publication No. CN 101098659. Concerning the glued endoscope front-end piece, due to characteristics of the glue, both reliability and durability of the product are greatly reduced, and there is an obvious mounting gap.

### SUMMARY

An objective of the present disclosure is to provide a mounting structure for an endoscope front-end piece, which realizes gapless mounting of the front-end piece. Without a structural force on a sealant, the present disclosure improves the service life and reliability, reduces the number of parts, and lowers the cost.

The present disclosure adopts the technical solutions as follows:

A mounting structure for an endoscope front-end piece includes a front-end piece, a pull rod, and an adjustment mechanism, where the front-end piece includes a front end clamped onto a port of an endoscope tube, and a rear end extended into the endoscope tube; a sealant groove is formed in a portion of the front-end piece contacting an inner wall of the endoscope tube; the pull rod includes a front end connected to the front-end piece, and a rear end extended into an endoscope tube socket; and the adjustment mechanism is located in the endoscope tube socket, and is capable of both compressing the endoscope tube socket and tensioning the pull rod through threaded transmission, thereby allowing the front-end piece to retain on the port of the endoscope tube, and adjusting a mounting gap and a mounting pressure.

As a preferable limitation, the adjustment mechanism may include an adjusting screw, an adjusting nut, and an opening gasket; a rear portion of the pull rod may be provided with a widened portion; the adjusting screw may be sleeved on the pull rod after passing through the widened portion; the adjusting nut may be threadedly engaged on the adjusting screw and may abut against an interior of the endoscope tube socket; the opening gasket may be provided at a rear end of the adjusting screw after placed onto the pull rod; and when the adjusting nut is tightened up, the adjusting screw can be driven through threaded transmission to move backward and to compress the widened portion through the opening gasket.

Further, the widened portion may be located at two sides of the rear portion of the pull rod, and formed into a T shape structure with the pull rod.

As another preferable limitation, the adjustment mechanism may include an adjustment fixing member and an adjusting bolt; a connecting portion may be defined on a rear portion of the pull rod; the adjustment fixing member may abut against an interior of the endoscope tube socket, and the connecting portion may be received in a front end of the adjustment fixing member; the adjusting bolt may be extended in threaded fit from a rear side of the adjustment fixing member, and may be in threaded fit with the connecting portion; and when the adjusting bolt is tightened up, the connecting portion can be driven through threaded transmission to move backward.

As a preferable limitation, the front portion of the pull rod may be provided with an elbow; and the elbow may be firmly hooked to a corresponding connecting groove defined in the front-end piece.

Further, there may be two elbows that may be distributed symmetrically.

As another preferable limitation, the front portion of the pull rod may be attached to the front-end piece and connected to the front-end piece through a locating bolt.

Further, the pull rod may be an integral part or a plurality of spliced parts.

Further, the sealant groove may be close to the front end of the front-end piece.

Further, a locating and mounting structure for a functional component may be provided on the front-end piece, and the functional component may include a camera module and an illumination module.

The present disclosure has the following beneficial effects:

Since the front-end piece is retained on the port of the endoscope tube through the pull rod, the structure realizes gapless mounting of the front-end piece. The sealant is only used for sealing rather than bearing a structural force, the present disclosure improves the service life and reliability, increases the mounting precision, reduces the number of parts, and lowers the cost.

### BRIEF DESCRIPTION OF THE DRAWINGS

FIG. 1 is a longitudinal sectional view of an endoscope according to Embodiment 1 of the present disclosure;

FIG. 2 is a first partially enlarged view of FIG. 1;

FIG. 3 is a second partially enlarged view of FIG. 1; and

FIG. 4 is a schematic view of a front-end piece in FIG. 1.

In the figures: 11. front-end piece, 111. locating and mounting hole for functional component, 112. sealant groove, 113. connecting groove, 114. locating step, 12. functional component, 13. endoscope tube, 14. pull rod, 141. widened portion, 142. elbow, 15. adjusting screw, 16. opening gasket, 17. adjusting nut, and 18. endoscope tube socket.

FIG. 5 is a longitudinal sectional view of an endoscope according to Embodiment 2 of the present disclosure;

FIG. 6 is a first partially enlarged view of FIG. 5;

FIG. 7 is a second partially enlarged view of FIG. 5;

FIG. 8 is a schematic view of a front-end piece in FIG. 5;

FIG. 9 is a schematic view of an adjustment fixing member in FIG. 5;

FIG. 10 is a schematic view of an adjusting segment of a pull rod in FIG. 5; and

FIG. 11 is another sectional view of the endoscope according to Embodiment 2 of the present disclosure.

In the figures: 21. front-end piece, 211. structure for locating and mounting functional component, 212. sealant groove, 213. connecting hole, 214. locating step, 22. functional component, 23. endoscope tube, 241. connecting segment of pull rod, 242. intermediate segment of pull rod, 243. adjusting segment of pull rod, 2431. adjusting bolt hole, 2432. connecting portion, 25. endoscope tube socket, 26. adjustment fixing member, 261. locating surface, 262. adjusting bolt hole, and 27. locating bolt, 28. adjusting bolt.

### DETAILED DESCRIPTION OF THE EMBODIMENTS

The present disclosure is further described below with reference to accompanying drawings and embodiments.

### Embodiment 1

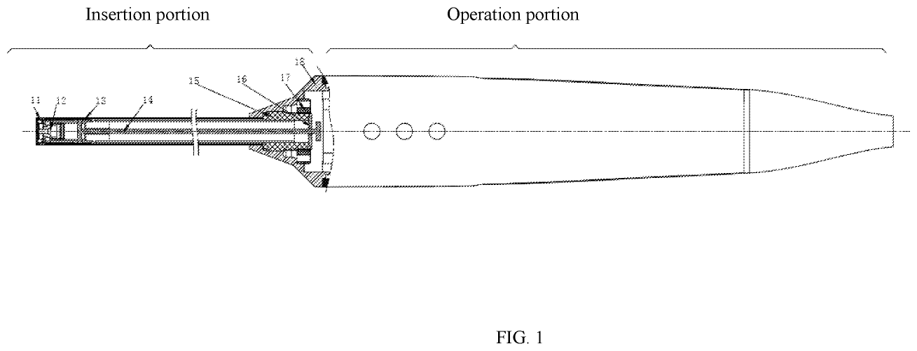

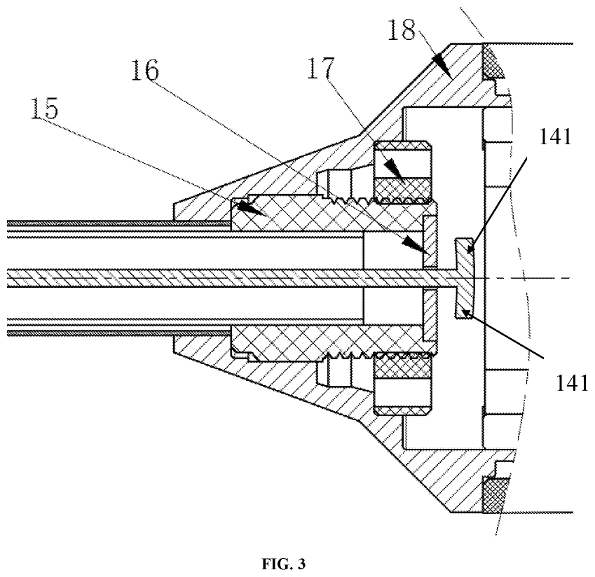

As shown in FIG. 1 to FIG. 4, a mounting structure for an endoscope front-end piece includes a front-end piece 11, a pull rod 14, and an adjustment mechanism. The front-end piece 11 includes a front end clamped onto a port of an endoscope tube 13 through a locating step 114, and a rear end extended into the endoscope tube 13. A sealant groove 112 is formed in a portion of the front-end piece 11 contacting an inner wall of the endoscope tube 13. The pull rod 14 is an integral part. The pull rod 14 includes a front end connected to the front-end piece 11, and a rear end extended into an endoscope tube socket 18. The adjustment mechanism is located in the endoscope tube socket 18, and is capable of both compressing the endoscope tube socket 18 and tensioning the pull rod 14 through threaded transmission, thereby allowing the front-end piece 11 to retain on the port of the endoscope tube 13, and adjusting a mounting gap and a mounting pressure. The endoscope tube 13 and the endoscope tube socket 18 are welded to form a main body of an insertion portion. Since the front-end piece 11 is retained on the port of the endoscope tube 13 through the pull rod 14, the structure realizes gapless mounting of the front-end piece 11. The sealant is only used for sealing rather than bearing a structural force, the present disclosure improves the service life and reliability, increases the mounting precision, reduces the number of parts, and lowers the cost.

As shown in FIG. 1 to FIG. 3, the adjustment mechanism in Embodiment 1 includes an adjusting screw 15, an adjusting nut 17, and an opening gasket 16. A rear portion of the pull rod 14 is provided with a widened portion 141. The widened portion 141 is located at two sides of the rear portion of the pull rod 14, and formed into a T shape structure with the pull rod 14, which ensures uniform stress and facilitates machining. The adjusting screw 15 is sleeved on the pull rod 14 after passing through the widened portion 141. The adjusting nut 17 is threadedly engaged on the adjusting screw 15 and abuts against an inner end surface of the endoscope tube socket 18. The opening gasket 16 is provided at a rear end of the adjusting screw 15 after placed onto the pull rod 14. When the adjusting nut 17 is tightened up, the adjusting screw 15 can be driven through threaded transmission to move backward and to compress the widened portion 141 through the opening gasket 16.

As shown in FIG. 1 to FIG. 3, in Embodiment 1, the front portion of the pull rod 14 is provided with elbows 142. The elbows 142 are firmly hooked to corresponding connecting grooves 113 in the front-end piece 11. There are two elbows 142 that may be distributed symmetrically, which ensures uniform stress, and facilitates machining.

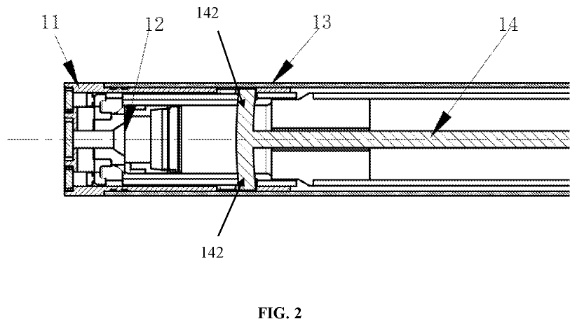

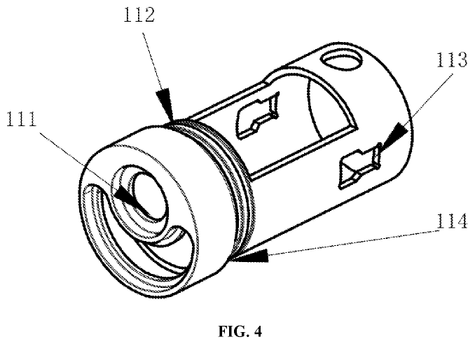

As shown in FIG. 2 and FIG. 4, in Embodiment 1, the sealant groove 112 is close to the front end of the front-end piece 11. There are two sealant grooves (certainly, there may be more sealant grooves).

As shown in FIG. 2 and FIG. 4, in Embodiment 1, a locating and mounting structure 111 for a functional component is provided on the front-end piece 11. The functional component 12 includes a camera module and an illumination module.

### Embodiment 2

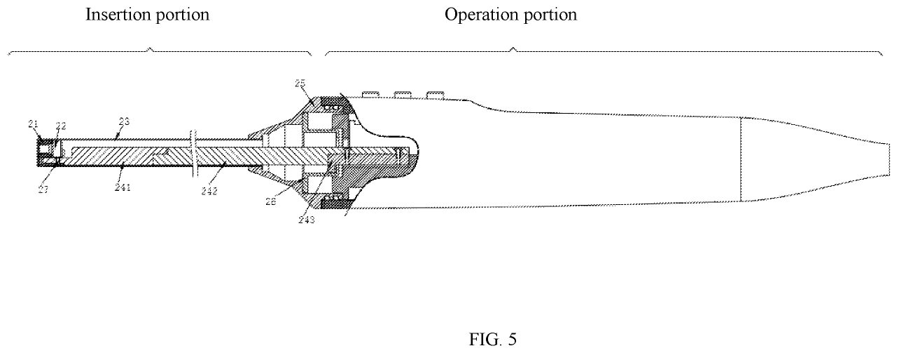

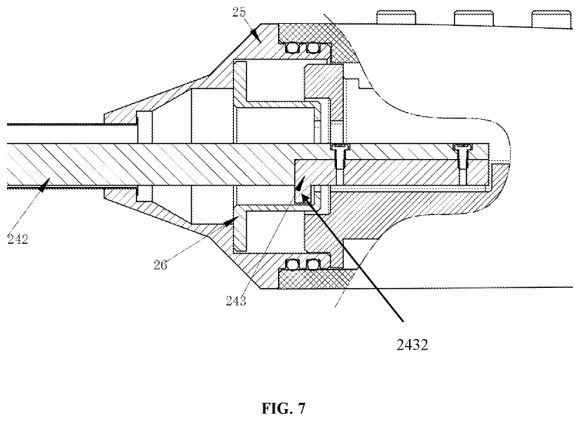

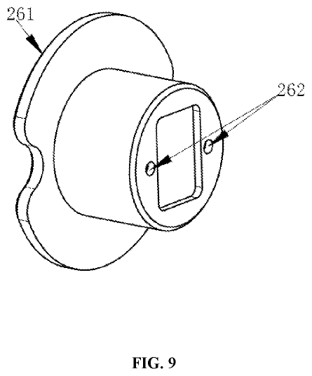

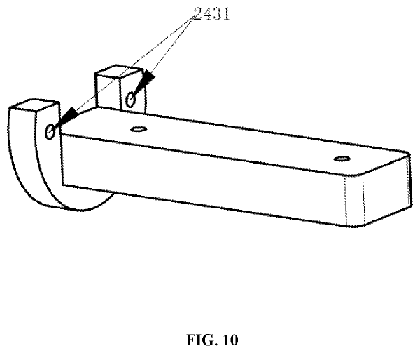

As shown in FIG. 5 to FIG. 10, a mounting structure for an endoscope front-end piece includes a front-end piece 21, a pull rod, and an adjustment mechanism. The front-end piece 21 includes a front end clamped onto a port of an endoscope tube 23 through a locating step 214, and a rear end extended into the endoscope tube 23. A sealant groove 212 is formed in a portion of the front-end piece 21 contacting an inner wall of the endoscope tube 23. The pull rod is spliced sequentially by a connecting segment 241, an intermediate segment 242, and an adjusting segment 243. The connecting segment 241 of the pull rod is connected to the front-end piece 21. The adjusting segment 243 of the pull rod is extended into an endoscope tube socket 25. The adjustment mechanism is located in the endoscope tube socket 25, and is capable of both compressing the endoscope tube socket 25 and tensioning the adjusting segment 243 of the pull rod through threaded transmission, thereby allowing the front-end piece 21 to retain on the port of the endoscope tube 23, and adjusting a mounting gap and a mounting pressure. The endoscope tube 23 and the endoscope tube socket 25 are welded to form a main body of an insertion portion. Since the front-end piece 21 is retained on the port of the endoscope tube 23 through the pull rod, the structure realizes gapless mounting of the front-end piece 21. The sealant is only used for sealing rather than bearing a structural force, the present disclosure improves the service life and reliability, increases the mounting precision, reduces the number of parts, and lowers the cost.

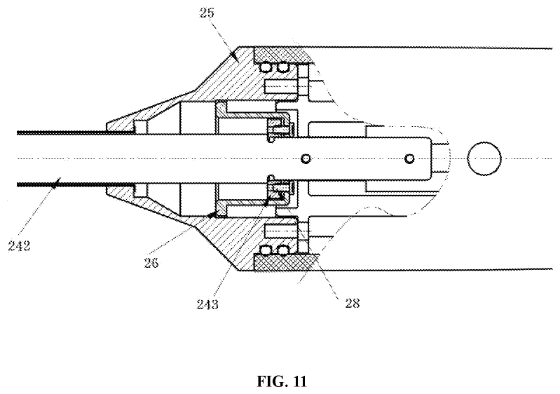

As shown in FIG. 5 to FIG. 7 and FIG. 11, the adjustment mechanism in Embodiment 2 includes an adjustment fixing member 26 and an adjusting bolt 28. A connecting portion 2432 is defined on the adjusting segment 243 of the pull rod. The adjustment fixing member 26 abuts against an interior of the endoscope tube socket 25, and with the connecting portion 2432 is received in a front end of the adjustment fixing member. The adjusting bolt 28 is extended in threaded fit from a rear side of the adjustment fixing member 26, and is in threaded fit with the connecting portion 2432. When the adjusting bolt 28 is tightened up, the connecting portion 2432 can be driven through threaded transmission to move backward.

As shown in FIG. 5 to FIG. 7, in Embodiment 2, the connecting segment 241 of the pull rod is attached to the front-end piece 21, and is connected to the front-end piece 21 through a locating bolt 27.

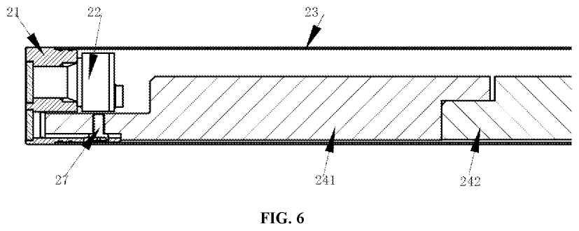

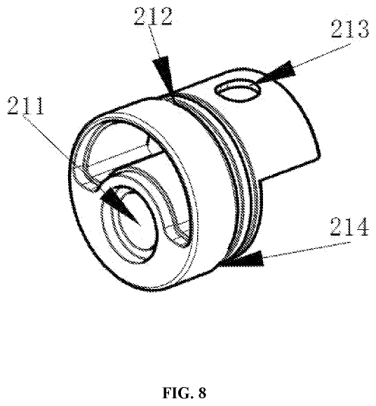

As shown in FIG. 6 and FIG. 8, in Embodiment 2, the sealant groove 212 is close to the front end of the front-end piece 21 (the number of the sealant grooves can be provided as required).

As shown in FIG. 6 and FIG. 8, in Embodiment 2, a structure 211 for locating and mounting a functional component is provided on the front-end piece 21. The functional component 22 includes a camera module and an illumination module.

The embodiments of the present disclosure have been described above with reference to the accompanying drawings, but the present disclosure is not limited to the foregoing specific embodiments. The foregoing specific embodiments are only illustrative and not restrictive. Under the inspiration of the present disclosure, those of ordinary skill in the art can make many improvements without departing from the purpose of the present disclosure and the protection scope defined by the claims, and these improvements shall fall within the protection scope of the present disclosure.

What is claimed is:

1. A mounting structure for an endoscope front-end piece, comprising a front-end piece, a pull rod, and an adjustment mechanism, wherein the front-end piece comprises a front end clamped onto a port of an endoscope tube, and a rear end extended into the endoscope tube; a sealant groove is formed in a portion of the front-end piece contacting an inner wall of the endoscope tube; the pull rod comprises a front end connected to the front-end piece, and a rear end extended into an endoscope tube socket; and the adjustment mechanism is located in the endoscope tube socket, and is capable of both compressing the endoscope tube socket and tensioning the pull rod through threaded transmission, thereby allowing the front-end piece to retain on the port of the endoscope tube, and adjusting a mounting gap and a mounting pressure.

2. The mounting structure according to claim 1, wherein the adjustment mechanism comprises an adjusting screw, an adjusting nut and an opening gasket; a rear portion of the pull rod is provided with a widened portion; the adjusting screw is sleeved on the pull rod after passing through the widened portion; the adjusting nut is threadedly engaged on the adjusting screw and abuts against an interior of the endoscope tube socket; the opening gasket is provided at a rear end of the adjusting screw after placed onto the pull rod; and when the adjusting nut is tightened up, the adjusting screw is driven through threaded transmission to move backward and to compress the widened portion through the opening gasket.

3. The mounting structure according to claim 2, wherein the widening portion is located at two sides of the rear portion of the pull rod, and formed into a T shape structure with the pull rod.

4. The mounting structure according to claim 1, wherein the adjustment mechanism comprises an adjustment fixing member and an adjusting bolt; a connecting portion is defined on a rear portion of the pull rod; the adjustment fixing member abuts against an interior of the endoscope tube socket, and the connecting portion is received in a front end of the adjustment fixing member; the adjusting bolt is extended in from a rear end of the adjustment fixing member, and is in threaded fit with the connecting portion; and when the adjusting bolt is tightened up, the connecting portion is driven through threaded transmission to move backward.

5. The mounting structure according to claim 1, wherein the front portion of the pull rod is provided with an elbow; and the elbow is firmly hooked to a corresponding connecting groove defined in the front-end piece.

6. The mounting structure according to claim 5, wherein there are two elbows that are distributed symmetrically.

7. The mounting structure according to claim 1, wherein the front portion of the pull rod is attached to the front-end piece and connected to the front-end piece through a locating bolt.

8. The mounting structure according to claim 1, wherein the pull rod is an integral part or a plurality of spliced parts.

9. The mounting structure according to claim 1, wherein the sealant groove is close to the front end of the front-end piece.

10. The mounting structure according to claim 1, wherein a structure for locating and mounting a functional component is defined on the front-end piece, and the functional component comprises a camera module and an illumination module.

11. The mounting structure according to claim 2, wherein the front portion of the pull rod is attached to the front-end piece and connected to the front-end piece through a locating bolt.

12. The mounting structure according to claim 3, wherein the front portion of the pull rod is attached to the front-end piece and connected to the front-end piece through a locating bolt.

13. The mounting structure according to claim 2, wherein the pull rod is an integral part or a plurality of spliced parts.

14. The mounting structure according to claim 3, wherein the pull rod is an integral part or a plurality of spliced parts.

15. The mounting structure according to claim 2, wherein the sealant groove is close to the front end of the front-end piece.

16. The mounting structure according to claim 3, wherein the sealant groove is close to the front end of the front-end piece.

17. The mounting structure according to claim 2, wherein a structure for locating and mounting a functional component is defined on the front-end piece, and the functional component comprises a camera module and an illumination module.

18. The mounting structure according to claim 3, wherein a structure for locating and mounting a functional component is defined on the front-end piece, and the functional component comprises a camera module and an illumination module.
